(19) United States
(12) Patent Application Publication
(10) Pub. No.: US 2013/0295145 A1
(43) Pub. Date: Nov. 7, 2013

(54) TOPICAL PHARMACEUTICAL COMPOSITIONS COMPRISING ETODOLAC

(71) Applicants: Sunilendu Bhushan Roy, Ahmedabad (IN); Jay Shantilal Kothari, Ahmedabad (IN); Shafiq Sheikh, Ahmedabad (IN); Jitendra Dasharathlal Patel, Ahmedabad (IN); Jinesh Suresh Pancholi, Ahmedabad (IN)

(72) Inventors: Sunilendu Bhushan Roy, Ahmedabad (IN); Jay Shantilal Kothari, Ahmedabad (IN); Shafiq Sheikh, Ahmedabad (IN); Jitendra Dasharathlal Patel, Ahmedabad (IN); Jinesh Suresh Pancholi, Ahmedabad (IN)

(73) Assignee: CADILA HEALTHCARE LIMITED, Ahmedabad, Gujarat (IN)

(21) Appl. No.: 13/880,302

(22) PCT Filed: Oct. 18, 2011
(86) PCT No.: PCT/IN11/00717

§ 371 Date: Jun. 21, 2013

(30) Foreign Application Priority Data
Oct. 21, 2010 (IN) 2925/MUM/2010

(51) Int. Cl. A61K 9/06; A61K 31/618; A61K 31/407

(52) U.S. Cl. CPC A61K 9/06; A61K 31/407; A61K 31/618
USPC 424/400; 514/411; 514/161

(57) ABSTRACT

The present invention relates to stable pharmaceutical compositions comprising nano size droplets of etodolac or salts thereof along with other pharmaceutically acceptable excipients. These compositions exhibit greater permeability, and improved bioavailability leading to enhanced therapeutic activity. The invention also relates to processes for the preparation of such compositions.

### FIELD OF THE INVENTION

The present invention relates to stable pharmaceutical compositions comprising nano size droplets of etodolac or salts thereof along with other pharmaceutically acceptable excipients. These compositions exhibit greater permeability, and improved bioavailability leading to enhanced therapeutic activity. The invention also relates to processes for the preparation of such compositions.

### BACKGROUND OF THE INVENTION

Pain can be defined as an unpleasant sensation ranging from mild discomfort to agonizing distress, associated with real or potential tissue damage, or a disorder of the nervous system. Pain is a response to impulses from the peripheral nerves in damaged tissue, which pass to nerves in the spinal cord. All animals experience some degree of pain during life, whether through injury or disease. As such, one of the major areas of drug research is the development of analgesics to be used in pain management.

One area in which pain is more frequently experienced than in others is inflammation. Pain associated with inflammation can be caused by pathologic processes in somatic structures or viscera or by prolonged dysfunction of parts the peripheral nervous system. Pain associated with inflammation may be the result of recurrent injuries, trauma, headache, arthritis including osteoarthritis, chronic obstructive pulmonary disease, psoriasis, or other pathologies. Pain associated with inflammation may be acute or chronic depending on the duration, level and extent of the inflammation.

Irrespective of the type or cause of pain it is important that early treatment is obtained as unrelieved pain can have profound psychological effects on the patient and acute pain which is poorly managed initially can degenerate into chronic pain which may prove more difficult to treat. However, the difficulty is that pain perception is a complex psychophysical process that can be modified by attitude, attention and suggestion. No other sensation depends as much on cognition and information processing as does pain.

Arthritis, a musculoskeletal disorder, is the leading cause of disability around the world. It is estimated that millions of people are now affected and expected to increase substantially by 2020.

Osteoporosis afflicts 200 million people worldwide and 25 million people in the U.S., of whom 80% are women, yet one man in three will also get osteoporosis. One woman in two over the age of 60 is likely to have an osteoporotic bone fracture. The incidence of hip fracture exceeds that of cancer of the breast, cervix and uterus combined. Sixteen percent of patients suffering hip fractures will die within six months, while 50% will require long-term nursing care. The estimated cost of treatment and care for osteoporosis and related fractures exceeds 10 billion per year in the U.S. alone.

Treatment usually consists of an oral ingestion of an analgesic such as nonsteroidal anti-inflammatory drugs (NSAIDs). However, use of NSAIDs is limited substantially due to their adverse side effects, such as hypersensitivity, gastropathy, renal impairment, liver toxicity and prolonged bleeding merit concern in the very young and elderly. The use of topical analgesic compositions to treat musculoskeletal disorders is an effort to overcome the side effects of oral preparations with the advantage of delivering the analgesic directly to the affected body part.

Nonsteroidal anti-inflammatory agents (NSAIDs) are useful in relieving pain and tissue swelling, chiefly by inhibiting the biosynthesis of prostaglandins. In small doses, NSAIDs have an analgesic action, but full doses have both analgesic and anti-inflammatory actions, and are effective in reducing pain and swelling. NSAIDs are the first choice for the management of pain.

Etodolac belongs to a class of drugs called nonsteroidal anti-inflammatory drugs (NSAIDs). It is marketed as oral immediate release tablets or capsules, extended release tablets and conventional topical formulations. Chemically, it is (RS)-2-(1,8-Diethyl-4,9-dihydro-3H-pyrano[3,4-b]indol-1-yl)acetic acid, having a structure of Formula I.

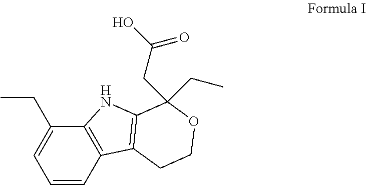 [SMILES: CCc1cccc2c3c([nH]c12)C(CC)(CC(=O)O)OCC3]

U.S. Pat. No. 6,399,093 discloses a method and composition for the treatment of musculoskeletal disorders in mammals by the application of a topical composition comprising a permeation enhancing amount of one or more penetration enhancers, and one or more bio-affecting agents to provide anti-inflammatory relief and analgesia to the applied body part.

U.S. Pat. No. 5,629,021 relates to micellar nanoparticles and methods of their production.

U.S. Pat. No. 5,894,019 discloses topical compositions comprising lipid and essentially free of emulsifiers and surfactants.

European Patent No. EP 506197 B1 discloses an aqueous suspension of solid lipid nanoparticles for topical use.

European Patent No. 671903 B1 discloses topical compositions in the form of submicron oil spheres.

Clinical evidence suggests that topically applied non-steroidal anti-inflammatory drugs (NSAIDs) are safer than and at least as efficacious as oral NSAIDs in the treatment of rheumatic diseases. Adverse drug reactions after topical administration of NSAID use are rare when compared to the incidence of serious GI events associated with oral NSAIDs. However, formulation may have a dramatic impact on depth of penetration at the site of application, retention of drug molecules within the layers of skin, concentrations achieved in the muscle tissue, synovial fluid and in systemic circulation.

Most of the topical preparations contain vehicles comprising permeation enhancers, solvents, and high amount of surfactants to achieve these goals. But use of these agents is harmful, especially in chronic application, as many of them are irritants.

Therefore, there exists a need to develop such topical preparations of etodolac which does not involve use of such agents as described above to facilitate drug permeation through the skin, and still leads to greater permeability, and improved bioavailability resulting in enhanced therapeutic activity.

The compositions of the invention overcome all the commonly encountered problems as exemplified above.

### SUMMARY OF THE INVENTION

In one general aspect there is provided a stable topical pharmaceutical composition of etodolac or salts thereof comprising nano size droplets of etodolac or salts thereof.

In another general aspect there is provided a stable topical pharmaceutical composition comprising combination of etodolac and methyl salicylate or salts thereof, wherein either etodolac or salts thereof, or both etodolac and methyl salicylate or salts thereof are present in the form of nano size droplets.

In another general aspect there is provided a stable topical pharmaceutical composition comprising nano size droplets of etodolac or salts thereof, wherein the amount of etodolac or salt thereof in the composition ranges from about 0.5% to about 3.0% w/w of the composition.

In another general aspect there is provided a stable topical pharmaceutical composition comprising nano size droplets of etodolac or salts thereof, wherein said composition comprises oil in amount ranging from about 5 to about 25% w/w of the composition.

In another general aspect there is provided a stable topical pharmaceutical composition comprising nano size droplets of etodolac or salts thereof, wherein said composition comprises one or more emulsifier/s in amount ranging from about 0.1 to about 10% w/w of the composition.

In another general aspect there is provided a stable topical pharmaceutical composition comprising nano size droplets of etodolac or salts thereof, wherein said composition comprises one or more emulsifier/s and oil in the weight ratio ranging from about 0.1:20 to about 0.1:1.

Embodiments of the stable topical pharmaceutical composition may include one or more of the following features. The pharmaceutical composition may further include one or more pharmaceutically acceptable excipients. For example, the pharmaceutically acceptable excipients may include one or more of oils, lipids, stabilizers, emulsifiers, pH adjusting agents, emollients, humectants, preservatives, stabilizers, antioxidants, chelating agents, initiators, thickening agents, and the like.

In another general aspect there is provided a stable topical pharmaceutical composition comprising nano size droplets of etodolac or salts thereof wherein the composition is characterized by enhanced onsite delivery.

In another general aspect there is provided a stable topical pharmaceutical composition comprising nano size droplets of etodolac or salts thereof, wherein the composition is characterized by enhanced onsite delivery, permeability characteristics and/or bioavailability.

In another general aspect there is provided a stable topical pharmaceutical composition of etodolac or salts thereof comprising nano size droplets of etodolac or salts thereof, wherein the composition retains at least 80% potency of etodolac or salts thereof after 3 months at 40° C. and 75% relative humidity.

Embodiments of the stable topical pharmaceutical composition may include one or more of the following features. The pharmaceutical composition may further include one or more pharmaceutically acceptable excipients. For example, the pharmaceutically acceptable excipients may include one or more of oils, lipids, stabilizers, emulsifiers, pH adjusting agents, emollients, humectants, preservatives, stabilizers, antioxidants, chelating agents, initiators, thickening agents, and the like.

D₉₀ particle size of droplets of etodolac or salts thereof in the compositions of the invention is less than 500 nm, preferably less than 200 nm, more preferably less than 100 nm.

In another general aspect there is provided a stable topical pharmaceutical composition of etodolac or salts thereof prepared by the process comprising:
a) combining an oily phase comprising etodolac or salts thereof along with other pharmaceutically acceptable excipients with an aqueous phase to form an emulsion;
b) reducing the particle size of emulsion of step a) to a droplet size having D₉₀ particle size of less than 500 nm; and
c) mixing other pharmaceutically acceptable excipients to the emulsion obtained in step b) and converting it into a suitable finished dosage form.

In another general aspect there is provided a method for management of pain comprising topical application of the pharmaceutical composition comprising nano size droplets of etodolac or salts thereof to the patient in need thereof.

Embodiments of the stable topical pharmaceutical composition may include one or more of the following features. The pharmaceutical composition may further include one or more pharmaceutically acceptable excipients. For example, the pharmaceutically acceptable excipients may include one or more of oils, lipids, stabilizers, emulsifiers, pH adjusting agents, emollients, humectants, preservatives, stabilizers, antioxidants, chelating agents, initiators, thickening agents, and the like.

The details of one or more embodiments of the invention are set forth in the description below. Other features, objects and advantages of the invention will be apparent from the description and claims.

### DETAILED DESCRIPTION OF THE INVENTION

The inventors of the invention have discovered that when etodolac or salts thereof is formulated into nano size droplets in pharmaceutically acceptable emulgel (emulsion gel) system which includes optimized ratios of oils and/or emulsifiers, the composition exhibits enhanced therapeutic effect and also the composition exhibits excellent storage stability. Further, such compositions have enhanced onsite delivery, enhanced permeability characteristics and/or improved bioavailability.

The composition of the invention results in immediate and sustained action and covers large surface area with less quantity and good spreadability, non-irritant to skin and mucous membranes, reduced frequency of application leading to improved patient compliance and offers cosmetic benefits like non-stickiness, and non-greasy feel. The embodiments of the present invention relate to a stable pharmaceutical composition comprising nano size droplets of etodolac or salts thereof.

In a preferred embodiment, the nano size droplets of etodolac or salts thereof posses a D₉₀ particle size of less than about 500 nm.

In a preferred embodiment, the nano size droplets of etodolac or salts thereof posses a D₉₀ particle size of less than about 400 nm.

In a preferred embodiment, the nano size droplets of etodolac or salts thereof posses a D₉₀ particle size of less than about 300 nm.

In a preferred embodiment, the nano size droplets of etodolac or salts thereof posses a D₉₀ particle size of less than about 200 nm.

In a preferred embodiment, the nano size droplets of etodolac or salts thereof posses a D₉₀ particle size of less than about 100 nm.

In a further embodiment, the composition of the present invention is stable and retains at least 80% potency of etodolac when stored for at least three months at 40° C. and 75% relative humidity.

The composition of the present invention may comprise combination of etodolac and one or more additional active agents selected from NSAIDs and methyl salicylate. The composition also retains at least 80% potency of additional active agents or salts thereof when stored for at least three months at 40° C. and 75% relative humidity.

Suitable NSAIDs which can be used include one or more of aspirin, benoxaprofen, benzofenac, bucloxic acid, butibufen, carprofen, cicloprofen, cinmetacin, clidanac, clopirac, diclofenac, fenbufen, fenclofenac, fenclorac, fenoprofen, fentiazac, flunoxaprofen, furaprofen, flurbiprofen, furobufen, furofenac, ibuprofen, ibufenac, indomethacin, indoprofen, isoxepac, ketoprofen, lactorolac, lonazolac, metiazinic, miroprofen, naproxen, oxaprozin, oxepinac, phenacitin, pirprofen, pirazolac, protizinic acid, sulindac, suprofen, tiaprofenic acid, tolmetin, zomepirac, ibuprofen, naproxen, benoxaprofen, flurbiprofen, fenoprofen, fenbufen, ketoprofen, indoprofen, pirprofen, carprofen, oxaprozin, pranoprofen, miroprofen, tioxaprofen, suprofen, alimoprofen, tiaprofenic acid, fluprofen, bucloxic acid, indomethacin, sulindac, tolmetin, zomepirac, alclofenac, ibufenac, isoxepac, furofenac, tiopinac, zidometacin, acemetacin, fentiazac, clidanac, oxpinac, mefenamic acid, meclofenamic acid, flufenamic acid, niflumic acid, tolfenamic acid, diflunisal, flufenisal, piroxicam, sudoxicam, isoxicam, celecoxib, veldecoxib and etoricoxib.

The invention further contemplates composition comprising nano size droplets of etodolac or its combination with one or more NSAIDs and/or methyl salicylate.

In an embodiment, the composition may comprise combination of etodolac and methyl salicylate or salts thereof, wherein either etodolac, or both etodolac and methyl salicylate are present in the form of nano size droplets.

The amount of etodolac or salt thereof in the composition may range from about 0.1% to about 10.0% w/w (based on 100% total weight of the composition).

The amount of methyl salicylate or salt thereof in the composition may range from about 12.0% to about 18.0% w/w (based on 100% total weight of the composition).

The composition of the present invention further comprises one or more pharmaceutically acceptable excipients selected from, but not limited to lipids, oils, emulsifiers, stabilizers, initiators, pH adjusting agents, emollients, humectants, preservatives, antioxidants and chelating agents.

Suitable lipids which can be used include one or more of hydrocarbons, fatty alcohols, fatty acids, glycerides or esters of fatty acids with C₁-C₃₆ alkanols. Hydrocarbons may include paraffin or petroleum jelly. Fatty alcohols may include decanol, dodecanol, tetradecanol, hexadecanol or octadecanol. Fatty acids may include C₆-C₂₄ alkanoic acids such as hexanoic acid, octanoic acid, decanoic acid, dodecanoic acid, tetradecanoic acid, hexadecanoic acid, octadecanoic acid, unsaturated fatty acids such as oleic acid and linoleic acid. Glycerides may include olive oil, castor oil, sesame oil, caprylic/capric acid triglyceride or glycerol mono-, di- and tri-esters with palmitic and/or stearic acid. Esters of fatty acids may include C₁-C₃₆ alkanols such as beeswax, carnauba wax, cetyl palmitate, lanolin, isopropyl myristate, isopropyl stearate, oleic acid decyl ester, ethyl oleate and C₆-C₁₂ alkanoic acid esters and the like.

Suitable oils may include one or more of almond oil, apricot seed oil, borage oil, canola oil, coconut oil, corn oil, cotton seed oil, fish oil, jojoba bean oil, lard oil, linseed oil, boiled macadamia nut oil, mineral oil, olive oil, peanut oil, safflower oil, sesame oil, soyabean oil, squalane, sunflower seed oil, tricaprylin (1,2,3 trioctanoyl glycerol) and wheat germ oil and the like. The preferred quantity of oil used is in the range of about 5 to about 25% w/w, and more preferably in the range of about 5% to about 20% w/w of the composition.

Suitable emulsifiers may include one or more of ionic polysorbate surfactant, Tween® 20, Tween® 40, Tween® 60, Tween® 80, Nonylphenol Polyethylene Glycol Ethers, (alkylphenol-hydroxypolyoxyethylene), Poly(oxy-1,2-ethanediyl), alpha-(4-nonylphenol)-omega-hydroxy-, branched (i.e. Tergitol® NP-40 Surfactant), Nonylphenol Polyethylene Glycol Ether mixtures (i.e. Tergitol® NP-70 (70% AQ) Surfactant), polymers or copolymers of acryilic acid (commercially available as Carbopol®), phenoxypolyethoxyethanols and polymers thereof such as Triton®, Poloxamer®, Spans®, Tyloxapol®, different grades of Brij, sodium dodecyl sulfate and the like. The preferred quantity of the emulsifiers used is in the range of about 0.1% to about 10% w/w of the composition.

In a preferred embodiment, the ratio of emulsifier or surfactant to oil in the pharmaceutical composition of the present invention ranges from about 0.1:20 to about 0.1:1, preferably about 0.1:10 to about 0.1:1.

Suitable pH adjusting agents which can be used include one or more of organic or inorganic acids and bases including sodium hydroxide, potassium hydroxide, ammonium hydroxide, phosphate buffers, citric acid, acetic acid, fumaric acid, hydrochloric acid, malic acid, nitric acid, phosphoric acid, propionic acid, sulfuric acid, tartaric acid and the like. In an embodiment, the pH of the composition of the invention may range from about 4.5 to about 7.0, and preferably from 5.0 to about 6.5.

Suitable emollients which can be used include one or more of caprylic/capric triglyerides, castor oil, ceteareth-20, ceteareth-30, cetearyl alcohol, ceteth 20, cetostearyl alcohol, cetyl alcohol, cetyl stearyl alcohol, cocoa butter, diisopropyl adipate, glycerin, glyceryl monooleate, glyceryl monostearate, glyceryl stearate, isopropyl myristate, isopropyl palmitate, lanolin, lanolin alcohol, hydrogenated lanolin, liquid paraffins, linoleic acid, mineral oil, oleic acid, white petrolatum, polyethylene glycol, polyoxyethylene glycol fatty alcohol ethers, polyoxypropylene 15-stearyl ether, propylene glycol stearate, squalane, steareth-2 or -100, stearic acid, stearyl alcohol, urea and the like.

Suitable preservatives which can be used include one or more of phenoxyethanol, parabens (such as methylparaben and propylparaben), propylene glycols, sorbates, urea derivatives (such as diazolindinyl urea), and the like.

Suitable antioxidants which can be used include one or more of ascorbic acid, alpha-tocopherol (vitamin-E), butylated hydroxyanisole, butylated hydroxytoluene, glutathione, sodium metabisulphite and the like.

Suitable humectants which can be used include one or more of propylene glycol, glycerin, butylene glycol, sorbitol, triacetin and the like.

Suitable chelating agents which can be used include one or more of disodium EDTA, edetate trisodium, edetate tetrasodium, diethyleneamine pentaacetate and the like.

Suitable stabilizers may include one or more of ionic polysorbate surfactant, Tween® 20, Twee' 40, Tween® 60, Tween® 80, Nonylphenol Polyethylene Glycol Ethers, (alkylphenol-hydroxypolyoxyethylene), Poly(oxy-1,2-ethanediyl), alpha-(4-nonylphenol)-omega-hydroxy-, branched (i.e. Tergitor NP-40 Surfactant), Nonylphenol Polyethylene Glycol Ether mixtures (i.e. Tergitol® NP-70 (70% AQ) Surfactant), phenoxypolyethoxyethanols and polymers thereof such as Triton®, Poloxamer®, Spans®, Tyloxapol®, different grades of Brij, sodium dodecyl sulfate and the like. The preferred quantity of the stabilizer or surfactant used is in the range of 1 to 10% w/w of the composition.

Suitable initiators may include one or more of alcohols like C₁-C₁₂ alcohols, diols and triols, glycerol, methanol, ethanol, propanol, octanol, and the like. The amount of initiator may range from about 3.0% to about 7.0% w/w of the total weight of the composition.

The composition of the invention may be prepared by a) combining an oily phase comprising etodolac or salts thereof along with other pharmaceutically acceptable excipients with an aqueous phase to form an emulsion; b) reducing the particle size of emulsion of step a) to a droplet size having D₉₀ particle size of 500 nm; and c) mixing other pharmaceutically acceptable excipients to emulsion obtained in step b) and converting it into a suitable finished dosage form.

In an embodiment, the process of preparing a stable pharmaceutical composition comprising nano size droplets of etodolac or salts thereof comprising:
a) preparing a hydroalcoholic phase of etodolac or salts thereof with one or more alcohol, emulsifier and thickening agent.
b) mixing the above hydroalcoholic phase was mixed with one or more oil and water.
c) homogenizing the blend of step (b) to reduce the droplet size to D₉₀ particle size of less than 500 nm to form a nano emulsion; and
   optionally adding the aqueous dispersion of thickening agent to the above nano emulsion to get the nanogel.

The nano size droplets may be produced with reciprocating syringe instrumentation, continuous flow instrumentation, high speed mixing or high pressure homogenization. However, it will appreciated to the person skilled in the art any known method of reducing the size of droplet may be adopted to serve the purpose of the present invention.

Small droplets of the nano emulsion may be formed by passing the emulsion through a homogeniser under different pressures ranging from 3,500-21,500 psi. The emulsion may be passed between 4-5 times under the same conditions to get a final D₉₀ droplet size of about 500 nm. The nano droplets formed may be filtered through 0.2 to 0.4 micron filter.

The gel base may be used in the present invention to form a gel matrix for the preparation of nanogel from nanoemulsion. The gel base comprises of one or more of thickening agents.

Suitable thickening agents may include one or more of cellulose polymer, a carbomer polymer, a carbomer derivative, a cellulose derivative, polyvinyl alcohol, poloxamers, polysaccharides and the like.

Suitable dosage form of the invention may include cream, gel, ointment, lotion, and emulsion.

In a preferred embodiment, the composition of the invention is in the form of gel.

The present invention further provides a method for management of pain comprising topical application of the pharmaceutical composition comprising nano size droplets of etodolac or salts thereof to the patient in need thereof.

The invention is further illustrated by the following examples which are provided to be exemplary of the invention and do not limit the scope of the invention.

While the present invention has been described in terms of its specific embodiments, certain modifications and equivalents will be apparent to those skilled in the art and are intended to be included within the scope of the present invention.

### EXAMPLE 1

### Etodolac Nanogel

TABLE 1

| | Formulation 1 | Formulation 2 |
|---|---|---|
| Ingredients | % w/w | % w/w |
| Etodolac | 0.5-3 | 0.5-3 |
| L-Arginine | — | 0.1-1 |
| Alcohol | 3-7 | — |
| Polysorbate 80 | 1-5 | 1-5 |
| Glycerol | 3-7 | 3-7 |
| Light Liquid paraffin | — | 7-12 |
| Soyben Oil | 7-12 | — |
| Methyl Salicylate | 12-18 | 12-18 |
| Menthol | 3-7 | 3-7 |
| Carbopol Ultrez 10 | 0.5-3 | 0.5-3 |
| Sodium Hydroxide | Q.S | Q.S |
| Purified Water | Q.S | Q.S |

Procedure: Etodolac, alcohol were dissolved with tween 80, glycerol, soyabean oil or light liquid paraffin, and aqueous L-Arginine solution. The resulting blend was homogenized to reduce the droplet size to D₉₀ particle size of about 250 nm by high pressure homogenization to get the nano emulsion. Desired pH of the nano emulsion was adjusted with sodium hydroxide solution. Finally, the aqueous dispersion of carbomer was mixed with nano emulsion to get nanogel.

### EXAMPLE 2

### Stability Study on Nanogel Composition of Example 1

TABLE 2

| | % Drug in the formulation | | | |
|---|---|---|---|---|
| Drug/Property | Initial | 1 Month | 2 Month | 3 Month |
| Etodolac | 101.6 | 99.5 | 97.7 | 95.9 |
| Methyl salicylate | 97.3 | 100.9 | 95.2 | 93.2 |
| pH | 5.96 | 5.86 | 5.45 | 5.83 |

Table 2 provides stability data of nanogel composition of Formulation 1 of Example 1 when stored at 40° C. and 75% relative humidity for three months and indicates that said compositions remains stable and retains at least 80% potency of etodolac and methyl salicylate over the storage period.

TABLE 3

| | % Drug in the formulation | | | |
|---|---|---|---|---|
| Drug/Property | Initial | 1 Month | 2 Month | 3 Month |
| Etodolac | 100.4 | 97.6 | 103.0 | 102.3 |
| Methyl salicylate | 100.2 | 101.2 | 97.7 | 97.2 |
| pH | 5.96 | 5.87 | 5.49 | 5.88 |

Table 3 provides stability data of nanogel composition of Formulation 2 of Example 1 when stored at 40° C. and 75% relative humidity for three months and indicates that said compositions remains stable and retains at least 80% potency of etodolac and methyl salicylate over the storage period.

While the invention has been described in terms of its specific embodiments, certain modifications and equivalents will be apparent to those skilled in the art and are intended to be included within the scope of the invention.

We claim:

1. A stable topical pharmaceutical composition comprising nano size droplets of etodolac or salts thereof.

2. The stable topical pharmaceutical composition of claim 1, wherein the nano size droplets of etodolac or salt thereof have a particle size (D₉₀) of about 500 nm or less.

3. The stable topical pharmaceutical composition of claim 1, wherein the nano size droplets of etodolac or salt thereof have a particle size (D₉₀) of about 300 nm or less.

4. The stable topical pharmaceutical composition of claim 1, wherein the nano size droplets of etodolac or salt thereof have a particle size (D₉₀) of about 100 nm or less.

5. The stable topical pharmaceutical composition of claim 1, wherein the composition comprises about 0.5% to about 3.0% w/w of etodolac or a salt thereof of the total weight of the composition.

6. The stable topical pharmaceutical composition of claim 1, wherein the composition further comprises methyl salicylate or salts thereof.

7. The stable topical pharmaceutical composition of claim 6, wherein the composition comprises about 12.0% to about 18.0% w/w of methyl salicylate or a salt thereof of the total weight of the composition.

8. The stable topical pharmaceutical composition of claim 1, wherein the composition further comprises nano size droplets of methyl salicylate or salts thereof.

9. The stable topical pharmaceutical composition of claim 1, wherein the composition further comprises one or more pharmaceutically acceptable excipients comprising lipids, oils, emulsifiers, initiators, pH adjusting agents, thickening agents, emollients, humectants, preservatives, antioxidants, and chelating agents.

10. The stable topical pharmaceutical composition of claim 9, wherein the emulsifiers comprise about 0.1% to about 10% w/w of the total weight of the composition.

11. The stable topical pharmaceutical composition of claim 9, wherein the oil comprises about 5% to about 25% w/w of the total weight of the composition.

12. The stable topical pharmaceutical composition of claim 9, wherein the emulsifiers and oil are present in the composition in a weight ratio of from about 0.1:20 to about 0.1:1.

13. The stable topical pharmaceutical composition of claim 1, wherein the composition retains at least 80% potency of etodolac or a salt thereof after storage for 3 months at 40° C. and 75% relative humidity.

14. The stable topical pharmaceutical composition of claim 1, wherein the composition is characterized by enhanced onsite delivery, permeability characteristics and/or bioavailability.

15. The stable topical pharmaceutical composition of claim 1, wherein the composition is in the form of a cream, an ointment, a gel, lotion, liniment, paste or an emulsion.

16. The stable topical pharmaceutical composition of claim 1, wherein the composition is in the form of a gel.

17. A topical gel comprising
(a) nano sized droplets comprising about 0.5% to about 3.0% w/w of etodolac or a salt thereof;
(b) optionally nano sized droplets comprising about 12.0% to about 18.0% w/w of methyl salicylate or a salt thereof;
(c) about 3.0% to about 7.0% w/w of alcohol;
(d) about 0.1% to about 10.0% w/w of emulsifiers;
(e) about 5% to about 25% w/w of oil/lipids;
(f) one or more pH adjusting agents; and
(g) water.

18. A process for the preparation of a stable topical pharmaceutical composition comprising etodolac or salts thereof, and one or more pharmaceutically acceptable excipients comprising one or more of lipids, oils, emulsifiers, initiators, pH adjusting agents, thickening agents, emollients, humectants, preservatives, and chelating agents, the process comprising:
a) combining an oily phase comprising one or more etodolac or salts thereof along with other pharmaceutically acceptable excipients with an aqueous phase to form an emulsion;
b) reducing the particle size of emulsion of step a) to a droplet size having a particle size (D₉₀) of about 500 nm or less; and
c) mixing the other pharmaceutically acceptable excipients to the emulsion obtained in step b) and converting it into a suitable finished dosage form.

19. A method for management of pain, the method comprising administering a stable topical pharmaceutical composition comprising nano size droplets of etodolac or salts thereof.

20. The method of claim 19, wherein the composition further comprises methyl salicylate or salts thereof.

21. The method of claim 19, wherein the composition further comprises nano size droplets of methyl salicylate or salts thereof.
